(19) United States
(12) Patent Application Publication
(10) Pub. No.: US 2003/0003581 A1
(43) Pub. Date: Jan. 2, 2003

(54) Method for targeting transcriptionally active loci

(76) Inventors: Aris N. Economides, Tarrytown, NY (US); Thomas M. DeChiara, Paramus, NJ (US); George D. Yancopoulos, Yorktown Heights, NY (US)

Correspondence Address: Linda O. Palladino, Regeneron Pharmaceuticals, Inc., 777 Old Saw Mill River Road, Tarrytown, NY, 10591, US

(21) Appl. No.: 10/163,196

(22) Filed: Jun. 5, 2002

Related U.S. Application Data
(60) Provisional application No. 60/296,260, filed on Jun. 6, 2001.

(51) Int. Cl. C12N 15/85; C12N 5/08

(52) U.S. Cl. 435/455; 435/366

(57) ABSTRACT

The present invention is a method of targeting promoter-less selection cassettes into transcriptionally active loci. In particular, the invention is a method for targeting promoter-less selection cassettes into transcriptionally active loci in stem cells or other eukaryotic cells with much greater efficiency than previously observed with other methods, thus reducing or eliminating the need to screen for targeted cells. The invention also encompasses the DNA targeting vectors, the targeted cells, as well as non-human organisms, especially mice, created from the targeted cells.

This application claims priority to U.S. Provisional Application No. 60/296,260, filed Jun. 6, 2001. Throughout this application various publications are referenced. The disclosures of these publications in their entireties are hereby incorporated by reference into this application.

### FIELD OF THE INVENTION

The field of this invention is a method of targeting promoter-less selection cassettes into transcriptionally active loci. In particular, the field of this invention is a method for targeting promoter-less selection cassettes into the ROSA26 locus in embryonic stem cells or other eukaryotic cells with much greater efficiency than previously observed with other methods. The field of the invention also encompasses the DNA targeting vectors, the targeted cells, as well as non-human organisms, especially mice, created from the targeted cells.

### BACKGROUND OF THE INVENTION

Transgenic and knockout (KO) animals are used extensively to gain insight into gene function and to evaluate putative drug-targets in whole organisms. In the case of KO animals, the gene of interest is usually replaced by a marker gene to create a heterozygous null allele which can then be bred to homozygocity (though a small number of knockout animals have hemizygous phenotypes due to haploinsufficiency (Lindsay et al., 2001, Nature, 410, 97-101; Nutt and Busslinger, 1999, Biol Chem, 380, 601-11; Nutt et al., 1999, Nat Genet, 21, 390-5; Schwabe et al., 2000, Am J Hum Genet, 67, 822-31; Wilkie, 1994, J Med Genet, 31, 89-98.) or imprinting (DeChiara et al., 1990, Nature, 345, 78-80). A homozygous null allele may lead to a phenotype that can be used to understand the function of the gene of interest in vivo. However, since about 60% of homozygous null allele mutant animals to do not exhibit a phenotype and, if they do exhibit a phenotype, the phenotype only supplies information as to what happens when the gene of interest is absent, a complimentary approach is often utilized in which a gene of interest is over-expressed and/or miss-expressed by the engineering of transgenic animals. In transgenic animals, depending on how the DNA construct or vector carrying the transgene is designed, the gene of interest can be over-expressed (i.e. expressed at levels higher that those normally produced by the wild type gene), miss-expressed (i.e. expressed in a tissue different from the tissue or tissues in which it is normally expressed and/or at a time that is not normally expressed), or both. Importantly, it should be noted that the expression levels and expression profiles depend to a large extent on the choice of promoter driving the transgene. Furthermore, transgenic animal technology can be used to express any conceivable version of the gene of interest, including mutant and tagged forms, without affecting the activity of the normal endogenous locus. Combined with the ability to turn expression of the transgene either on or off at specific points in time or under certain sets of conditions (for example, by using regulated Cre technology (Kellendonk et al., 1996, Nucleic Acids Res, 24, 1404-11; Nagy and Mar, 2001, Methods Mol Biol, 158, 95-106; Rossant and McMahon, 1999, Genes Dev, 13, 142-5; Schwenk et al., 1998, Nucleic Acids Res, 26, 1427-32; Vooijs et al., 2001, EMBO Rep, 2, 292-297.), Tet-regulated systems (Baron and Bujard, 2000, Methods Enzymol, 327,401-21; Blau and Rossi, 1999, Proc Natl Acad Sci USA, 96, 797-9.; Gossen and Bujard, 1992, Proc Natl Acad Sci USA, 89, 5547-51; Gossen et al., 1995, Science, 268, 1766-9; Shockett and Schatz, 1996, Proc Natl Acad Sci USA, 93, 5173-6), Tet-ER (Fandl, James, “Inducible Eukaryotic Expression System”, U.S. Provisional Application No. ______, filed May 29, 2002, in the name of Regeneron Pharmaceuticals, Inc., incorporated by reference herein in its entirety) or other suitable technology familiar in the art), it is possible to carefully dissect the in vivo functions of a gene of interest and to evaluate drug-target candidate genes and novel protein-based therapeutics.

In spite of the advantages and utility of transgenic animal technology, currently available methods for creating transgenic animals suffer from several technological problems. The most frequently utilized method for creating a transgenic mouse is pronuclear injection (Jackson and Abbot, 2000, The Practical Approach Series, 299). In this method, a DNA construct or vector carrying the gene of interest is inserted downstream of a promoter and is followed by a polyadenylation signal sequence. The promoter is generally chosen on the basis of its tissue specificity. In some instances, it is desirable to use a ubiquitous promoter (i.e. one that is expressed in many, if not all, the different tissues and cell types in the organism), whereas in other instances it is desirable to use a tissue specific promoter (i.e. one that is expressed in only one or a few tissues). Once constructed, the DNA construct or vector is injected into oocytes that are then implanted into foster mothers. Once founder pups are born they have to be screened for expression of the transgene. Some of the more serious problems associated with this method arise from the fact that the introduced DNA construct integrates randomly and frequently in multiple copies into the genome. In turn, this random integration can often lead to several subsequent problems that become apparent upon examination of founders such as:

A. Positional effects: Aberrant expression of the transgene (i.e. expression that does not reflect the choice of promoter) is frequently observed. This can result from integration within or near a locus that contains regulatory elements that specify expression in a tissue other than the tissue that the promoter used in construct is specific for. Positional effects are particularly a problem for creating transgenic animals wherein ubiquitous expression of the gene of interest is desired. Typically, to create such animals, expression of the gene is driven by a ubiquitous promoter. However, mirroring the situation described above, it is often found that integration of the DNA construct within or near a locus that contains regulatory elements restricts the expression of the gene of interest to only a subset of tissues. Although positional effects can be minimized by using BAC-based transgenic animal technologies (Jackson and Abbot, 2000, The Practical Approach Series, 299; Yang et al., 1997, Nat Biotechnol, 15, 859-65; Yang et al., 1999, Nat Genet, 22, 327-35), this method still has the problems described below and, in addition, since a single BAC may contain multiple genes, making a BAC-based transgenic animal can result in generating transgenic animals that express not only the gene of interest, but any neighboring gene that might reside on the same BAC.

(B) Silencing of the transgene: It has been observed that multiple integrations of the transgene can lead to silencing (Garrick et al., 1998, Nat Genet, 18, 56-9; Henikoff, 1998, Bioessays, 20, 532-5; Lau et al., 1999, Dev Dyn, 215, 126-38) and instability (Schmidt-Kastner et al., 1996, Somat Cell Mol Genet, 22, 383-92.). This effect can confound screening of founders (see below).

(C) Insertional inactivation of an endogenous allele: It has been reported in the literature that insertion of the DNA construct or vector can unintentionally inactivate or alter the expression pattern of an endogenous gene (Merlino et al., 1991, Genes Dev, 5, 1395-406). Although this may not be a problem if the transgenic animals are maintained as heterozygotes (assuming that there is no phenotype due to haplo-insufficiency), it can confound breeding steps. Furthermore, if the insertional inactivation is not detected it can confuse interpretation of a phenotype by attributing the phenotype to expression of the transgene when in fact it is due to the generation of a null for the gene where the transgenic DNA construct has inserted itself. It has been estimated that as many as 10% of random integrations result in insertional inactivation of genes located at the site of integration (Jackson and Abbot, 2000, The Practical Approach Series, 299). Such events are hard to discover prior to phenotypic analysis. Although one may characterize the site of the insertion by cloning sequences upstream and downstream of the transgene, it may be difficult to determine exactly where the transgene has integrated because the mouse genome has yet to be sequenced and annotated to completion. In addition, the integration event may disrupt a regulatory element. Identifying which regulatory elements and/or genes have been disrupted is extremely complicated and difficult to do.

Taken together, these problems result in an overall uncertainty of the phenotype of transgenic animals derived by this method. Because of the above-described problems, for each transgenic animal line created, at least several founder lines must be screened for the expression levels and profile of the transgene. Finally, even if a founder with the desirable expression level and profile is discovered, insertional inactivation of an endogenous locus may still be a problem.

Another method for creating transgenic animals utilizes embryonic stem (ES) cells (Pirity et al., 1998, Methods Cell Biol, 57, 279-93; Rossant et al., 1993, Philos Trans R Soc Lond B Biol Sci, 339, 207-15). Although this method does not rely on pronuclear injection, it does rely on random integration of the gene of interest and thus it also is susceptible to the problems described above. More recently, the idea of creating a transgenic animal by introducing the gene of interest into a specific chromosomal locus has been explored. Two different types of insertions have been made. The first method involves the introduction of a ‘promoter-gene of interest-polyadenylation site cassette’ into a specific chromosomal locus, such as the hprt locus (Evans et al., 2000, Physiol Genomics, 2, 67-75; Wallace et al., 2000, Nucleic Acids Res, 28, 1455-64). The disadvantage of this approach lies in the choice of the hprt locus for targeting. The hprt locus is subject to X-linked inactivation, and this complicates breeding steps, as female mice have to be bred to homozygocity for reliable transmission of a transcriptionally active transgene to their progeny. The second method involves the introduction of a gene of interest into a specific chromosomal locus, thus utilizing the regulatory elements of that locus to control gene expression. In this situation, expression of the gene of interest should be nearly identical to and, therefore, also limited to, that of the gene(s) expressed by the targeted locus. Note that this method leads to a heterozygous null for the gene(s) residing within the targeted endogenous locus and thus the locus must be carefully selected for lack of a hemizygous null phenotype (Lindsay et al., 2001, Nature, 410, 97-101; Nutt and Busslinger, 1999, Biol Chem, 380, 601-11; Nutt et al., 1999, Nat Genet, 21, 390-5.; Wilkie, 1994, J Med Genet, 31, 89-98). Moreover, special care should be taken in maintaining such transgenic lines as heterozygous carriers since breeding to homozygocity would lead to generation of a homozygous null at the locus where the transgene has been introduced, and thus may exhibit a phenotype unrelated to the presence of the transgene. Finally, the expression of the transgene would be limited to those tissues where the gene encoded by the unmodified locus is expressed. Thus, although this strategy is very useful for creating tissue-specific transgenics it is still limited to loci for which either no phenotype results from haploinsufficiency and possibly also limits the potential for breeding to homozygocity.

Although these methods solve the positional effect problems and bypass the need to screen for founders, they retain other problems such as complicated breeding steps or insertional inactivation of endogenous chromosomal loci that may be required for normal functioning of the animal. Therefore, a need still remains for methods that allow rapid, reproducible, efficient, and simple generation of transgenic and knockout animals that are devoid of the confounding issues that exist in currently available methods.

### SUMMARY OF THE INVENTION

In accordance with the present invention, Applicants provide a novel method of targeting promoter-less selection cassettes into transcriptionally active loci. In particular, the invention is a method for targeting promoter-less selection cassettes into the ROSA26 locus in eukaryotic cells, thus achieving much greater targeting efficiencies than those previously obtained with other methods and requiring considerably less effort in screening for correctly targeted events. The novel methods of the invention also overcome the problems associated with current methodologies such as insertional inactivation of endogenous chromosomal loci and positional effects on transgene expression.

The DNA targeting vectors of the subject invention utilize a selection strategy that relies on the expression of a positive drug selection marker that is driven by the endogenous promoter of a transcriptionally active locus that is being targeted. Transcriptionally active loci are loci that at the current state of differentiation of the cell are accessible to the transcriptional machinery, and message resulting from their transcription can be found inside the cell. By targeting a transcriptionally active locus using targeting vectors that do not carry a promoter to transcribe the drug selection marker and thus rely on the promoter residing at the locus being targeted for transcription of the drug selection marker, only targeted clones are effectively selected for. A non-limiting example of a transcriptionally active locus that has been utilized by Applicants in practicing the method of the invention is the ROSA26 locus. Other examples of transcriptionally active loci are the BT-5 locus (Michael et al, 1999 Mech Dev 85, 35-47) and Oct4 (Wallace, 2000 Nucleic Acids Res 28, 1455-64), each of which may be suitable for practicing the methods of the invention.

Using the ROSA26 locus as the representative transcriptionally active locus, Applicants have found that mostly targeted clones result, thus alleviating the need to screen for targeted clones by Southern blotting or other diagnostic methods familiar in the art. This makes possible the use of pools of targeted cells rather than individual cell clones for the generation of transgenic animals, thus eliminating the problems that are encountered when using individual clones such as the unintentional use of mutated clones to generate the chimeric animals, achieving a low degree of chimerism, and/or the lack of germline transmission.

In accordance with the present invention, Applicants describe herein a novel method to perform gene targeting with nearly 100% efficiency, i.e. where essentially all of the drug-resistant cells that arise from selection are correctly targeted and contain a homologous recombination-mediated integration of the targeting vector. This novel method combines for the first time:

(1) targeting into a transcriptionally active locus, with

(2) the use of a “promoter-less selection cassette” to effectively select for only those cells that are correctly targeted by utilizing a targeting vector that relies on the endogenous promoter of the locus being targeted for transcription of the drug selection gene. The ability to select for correctly targeted eukaryotic cells allows the use of targeted cell pools rather than individual targeted cell clones for generating transgenic animals. Additional advantages include (a) greatly reducing the need to screen for correctly targeted clones thus providing a savings of time, labor, and the associated costs and (b) reducing the probability of selecting cell clones that generate transgenic animals with a low degree of chimerism, transgenic animals that cannot contribute to the germ line, or transgenic animals that are otherwise mutated and may result in a phenotypic outcome unrelated to the expression of the transgene.

The following is a non-limiting summary of some of the preferred embodiments of the methods of the invention.

One preferred embodiment of the invention is a method of targeting a promoter-less selection cassette into the ROSA26 locus in eukaryotic cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into eukaryotic cells;

c) selecting the eukaryotic cells of (b) for drug-resistance, and

d) screening the drug-resistant eukaryotic cells of (c) to identify those cells in which the promoter-less selection cassette has integrated by homologous recombination into the ROSA26 locus.

Also preferred is a method of targeting a promoter-less selection cassette into the ROSA26 locus in stem cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into stem cells;

c) selecting the stem cells of (b) for drug-resistance, and

d) screening the drug-resistant stem cells of (c) to identify those cells in which the promoter-less selection cassette has integrated by homologous recombination into the ROSA26 locus.

An additional preferred embodiment of the invention is a method of targeting a promoter-less selection cassette into a ROSA26 locus in embryonic stem cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into embryonic stem cells;

c) selecting the embryonic stem cells of (b) for drug-resistance, and

d) screening the drug-resistant embryonic stem cells of (c) to identify those cells in which the promoter-less selection cassette has integrated by homologous recombination into the ROSA26 locus.

Another embodiment is a method of targeting a promoter-less selection cassette into a transcriptionally active locus in eukaryotic cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into eukaryotic cells;

c) selecting the eukaryotic cells of (b) for drug-resistance, and

d) screening the drug-resistant eukaryotic cells of (c) to identify those cells in which the promoter-less selection cassette has integrated by homologous recombination into the transcriptionally active locus.

Also preferred is a method of targeting a promoter-less selection cassette into a transcriptionally active locus in stem cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into stem cells;

c) selecting the stem cells of (b) for drug-resistance, and

d) screening the drug-resistant stem cells of (c) to identify those cells in which the promoter-less selection cassette has integrated by homologous recombination into the transcriptionally active locus.

An additional preferred embodiment of the invention is a method of targeting a promoter-less selection cassette into a transcriptionally active locus in embryonic stem cells, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into embryonic stem cells;

c) selecting the embryonic stem cells of (b) for drug-resistance, and

d) screening the drug-resistant embryonic stem cells of (c) to identify those cells in which the promoter-less selection cassette has integrated by homologous recombination into the transcriptionally active locus.

Yet another preferred embodiment is a method of genetically modifying a eukaryotic cell by targeting a promoter-less selection cassette into the ROSA26 locus, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into eukaryotic cells;

c) selecting the eukaryotic cells of (b) for drug-resistance, and

d) screening the drug-resistant eukaryotic cells of (c) to identify those cells in which the promoter-less selection cassette has integrated by homologous recombination into the ROSA26 locus.

An additional preferred embodiment is a method of genetically modifying a stem cell by targeting a promoter-less selection cassette into the ROSA26 locus:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into stem cells;

c) selecting the stem cells of (b) for drug-resistance, and

d) screening the drug-resistant stem cells of (c) to identify those cells in which the promoter-less selection cassette has integrated by homologous recombination into the ROSA26 locus.

One embodiment is a method of genetically modifying an embryonic stem cell by targeting a promoter-less selection cassette into a ROSA26 locus, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into embryonic stem cells;

c) selecting the embryonic stem cells of (b) for drug-resistance, and

d) screening the drug-resistant embryonic stem cells of (c) to identify those cells in which the promoter-less selection cassette has integrated by homologous recombination into the ROSA26 locus.

Another embodiment is a method of genetically modifying a eukaryotic cell by targeting a promoter-less selection cassette into a transcriptionally active locus, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into eukaryotic cells;

c) selecting the eukaryotic cells of (b) for drug-resistance, and

d) screening the drug-resistant eukaryotic cells of (c) to identify those cells in which the promoter-less selection cassette has integrated by homologous recombination into the transcriptionally active locus.

An additional embodiment is a method of genetically modifying a stem cell by targeting a promoter-less selection cassette into a transcriptionally active locus, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into stem cells;

c) selecting the stem cells of (b) for drug-resistance, and

d) screening the drug-resistant stem cells of (c) to identify those cells in which the promoter-less selection cassette has integrated by homologous recombination into the transcriptionally active locus.

A preferred embodiment of the invention is a method of genetically modifying an embryonic stem cell by targeting a promoter-less selection cassette into a transcriptionally active locus, comprising:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into embryonic stem cells;

c) selecting the embryonic stem cells of (b) for drug-resistance, and

d) screening the drug-resistant embryonic stem cells of (c) to identify those cells in which the promoter-less selection cassette has integrated by homologous recombination into the transcriptionally active locus.

An additional preferred embodiment is a non-human organism containing a genetically modified ROSA26 locus, produced by a method comprising the steps of:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the ROSA26 locus;

b) introducing the DNA targeting vector of (a) into eukaryotic cells;

c) selecting the eukaryotic cells of (b) for drug-resistance,

d) screening the drug-resistant eukaryotic cells of (c) to identify those cells in which the promoter-less selection cassette has integrated by homologous recombination into the ROSA26 locus,

e) introducing the eukaryotic cells of (d) into a blastocyst; and

f) introducing the blastocyst of (e) into a surrogate mother for gestation.

Also preferred is a non-human organism containing a genetically modified transcriptionally active locus, produced by a method comprising the steps of:

a) constructing a DNA targeting vector containing a nucleotide sequence, comprising:

a 5′ homology arm,

a promoter-less selection cassette, and

a 3′ homology arm,

wherein the promoter-less selection cassette is comprised of a promoter-less selectable marker gene, a gene of interest, and a polyadenylation signal sequence and wherein the 5′ and 3′ homology arms are derived from the transcriptionally active locus;

b) introducing the DNA targeting vector of (a) into eukaryotic cells;

c) selecting the eukaryotic cells of (b) for drug-resistance,

d) screening the drug-resistant eukaryotic cells of (c) to identify those cells in which the promoter-less selection cassette has integrated by homologous recombination into the transcriptionally active locus,

e) fusing the eukaryotic cell of (d) with another eukaryotic cell; and

f) introducing the fused eukaryotic cell of (e) into a surrogate mother for gestation.

Other embodiments are where the genetic modification to the transcriptionally active locus comprises deletion of a coding sequence, gene segment, or regulatory element; alteration of a coding sequence, gene segment, or regulatory element; insertion of a new coding sequence, gene segment, or regulatory element; creation of a conditional allele; or replacement of a coding sequence or gene segment from one species with an homologous or orthologous coding sequence from the same or a different species, and in particular wherein the alteration of a coding sequence, gene segment, or regulatory element comprises a substitution, addition, or fusion., wherein the fusion comprises an epitope tag or bifunctional protein.

In another preferred embodiment of the invention the embryonic stem cell is a mouse, rat, or other rodent embryonic stem cell.

Other preferred embodiments are where the blastocyst is a mouse, rat, or other rodent blastocyst and the surrogate mother is a mouse, rat, or other rodent.

In a preferred embodiment the non-human organism is a mouse.

### DEFINITIONS

“Transgenic” cell or transgenic organism means a cell or organism that has been genetically altered so as to express a gene in a manner that is not normally expressed in that cell or organism.

“Promoter-less” means lacking a promoter that can confer expression in eukaryotic cells.

“Promoter-less selection cassette” is a DNA cassette containing a selectable marker gene(s) or cDNA(s) that lacks a mammalian promoter. The cassette may contain other genetic elements that do not cause expression of the selectable marker gene(s) or cDNA(s).

“Transcriptionally active loci” are loci that at the current state of differentiation of the cell are accessible to the transcriptional machinery, and message resulting from their transcription can be found inside the cell.

A “targeting vector” is a DNA construct that contains sequences “homologous” to endogenous chromosomal nucleic acid sequences flanking a desired genetic modification(s). The flanking homology sequences, referred to as “homology arms”, direct the targeting vector to a specific chromosomal location within the genome by virtue of the homology that exists between the homology arms and the corresponding endogenous sequence and introduce the desired genetic modification by a process referred to as “homologous recombination”.

“Homologous” means two or more nucleic acid sequences that are either identical or similar enough that they are able to hybridize to each other or undergo intermolecular exchange.

“Gene targeting” is the modification of an endogenous chromosomal locus by the insertion into, deletion of, or replacement of the endogenous sequence via homologous recombination using a targeting vector.

A “transgenic” cell or organism is a cell or organism into which a gene(s) or genetic locus or loci have been introduced into its genome.

A “gene knock-out” is a genetic modification resulting from the disruption of the genetic information encoded in a chromosomal locus.

A “gene knock-in” is a genetic modification resulting from the replacement of the genetic information encoded in a chromosomal locus with a different DNA sequence.

A “knock-out organism” is an organism in which a significant proportion of the organism's cells harbor a gene knock-out.

A “knock-in organism” is an organism in which a significant proportion of the organism's cells harbor a gene knock-in.

A “marker” or a “selectable marker” is a selection marker that allows for the isolation of rare transfected cells expressing the marker from the majority of treated cells in the population. Such marker's gene's include, but are not limited to, neomycin phosphotransferase and hygromycin B phosphotransferase, or fluorescing proteins such as GFP.

An “ES cell” is an embryonic stem cell. This cell is usually derived from the inner cell mass of a blastocyst-stage embryo.

An “ES cell clone” is a subpopulation of cells derived from a single cell of the ES cell population following introduction of DNA and subsequent selection.

A “flanking DNA” is a segment of DNA that is collinear with and adjacent to a particular point of reference.

A “non-human organism” is an organism that is not normally accepted by the public as being human.

“Orthologous” sequence refers to a sequence from one species that is the functional equivalent of that sequence in another species.

The description and examples presented infra are provided to illustrate the subject invention. One of skill in the art will recognize that these examples are provided by way of illustration only and are not included for the purpose of limiting the invention.

### BRIEF DESCRIPTION OF THE FIGURES

FIG. 1: A typical selection marker gene-containing cassette consists of a ubiquitously expressed promoter such as the phosphoglycerate kinase promoter (pgk), which drives the expression of a positive drug selection gene such as neomycin phosphotransferase or other suitable drug selection, followed by a polyadenylation signal sequence.

FIGS. 2A and 2B: A comparison of a traditional targeting vector (FIG. 2A) and a promoter-less selection cassette-containing targeting vector (FIG. 2B).

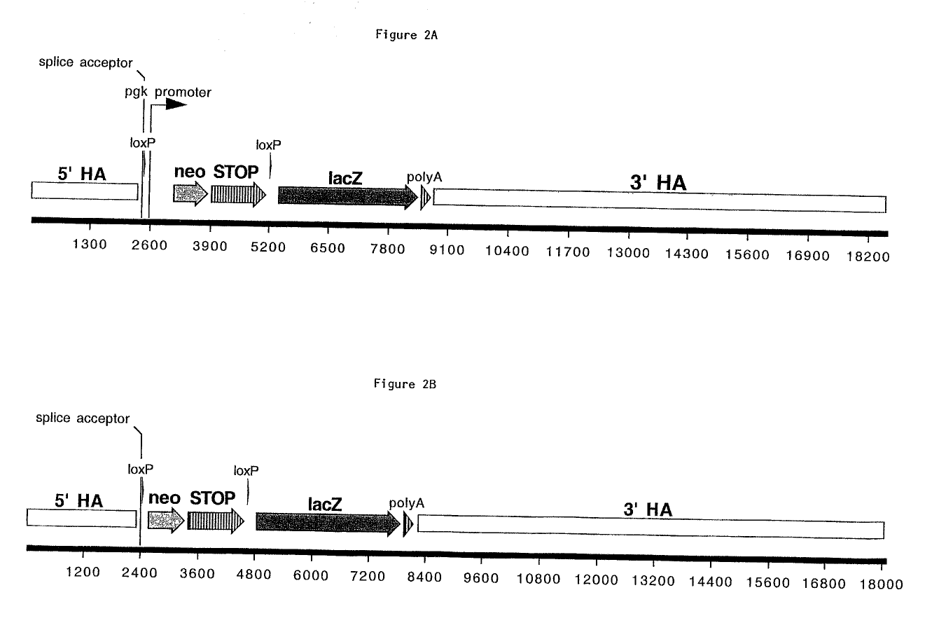

FIG. 3: A schematic representation of a typical DNA targeting vector. The vector contains a 5′ homology arm which contains sequence downstream of exon 1 of the ROSA26 locus; a promoter-less selection cassette containing SA-loxP-EM7-neo-4xpolyA-loxP, wherein SA is a splice acceptor sequence, the two loxP sites are the locus of recombination sites derived from bacteriophage P1, the neomycin (neo) phosphotransferase gene, and 4xpolyA which is a polyadenylation signal engineered by linking in tandem the polyadenylation signal of the murine pgk gene and three copies of a 254 bp BamHI fragment containing both early and late polyadenylation signals of Simian Virus 40 (SV40). After the second loxP site, a LacZ ORF has been engineered, followed by a human β-globin polyA. The β-globin polyA is followed by a 3′ homology arm containing sequence continuous to that of the 5′ homology arm.

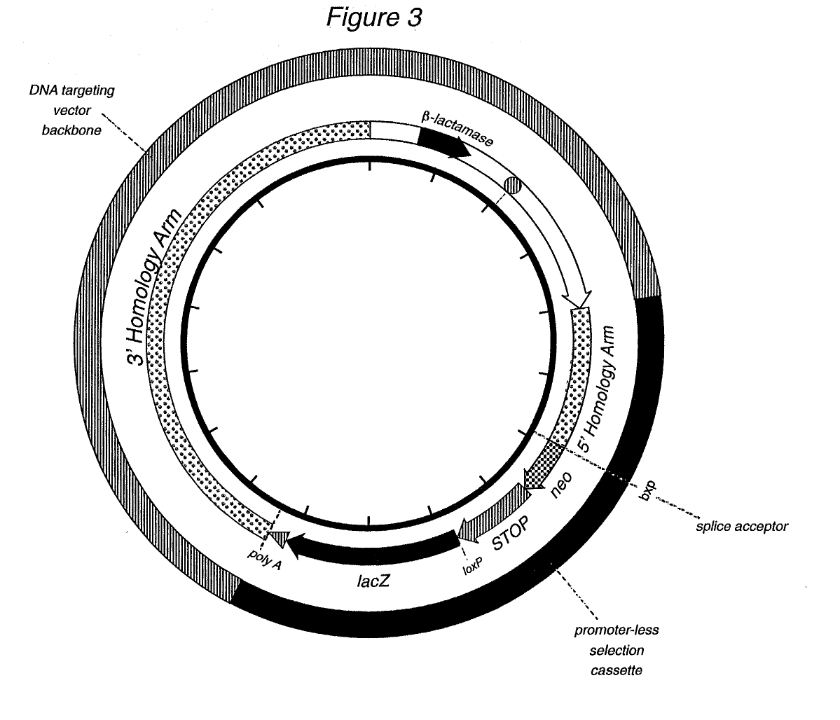

### DETAILED DESCRIPTION OF THE INVENTION

Currently available methods for generating ES cells useful for creating genetically modified mammals include pronuclear injection, or using modified ES cells. The methods utilizing pronuclear injection of DNA constructs or vectors containing sequences encoding a promoter, the gene of interest, a polyadenylation sequence and other regulatory or accessory elements, has been widely used but suffers from several serious drawbacks that arise primarily from the fact that the transgene is integrated randomly into the genome. These drawbacks are outlined in detail in the Background section supra. Some of the methods that utilize ES cells have also relied on random integration of the transgene, though more recently the idea of targeting the transgenic construct into specific chromosomal loci has also been utilized. Although the latter method provides solutions to some of the problems encountered with methods where the transgene is integrated randomly, these methods still rely on gene targeting technology that gives rise to a high number of non-targeted (and therefore not useful) versus targeted ES cell clones. Applicants, therefore, describe herein a new and novel method to perform gene targeting wherein virtually all the cells that survive drug selection arise from a correctly targeted event, thus eliminating, in most instances, the need for extensive screening of clones.

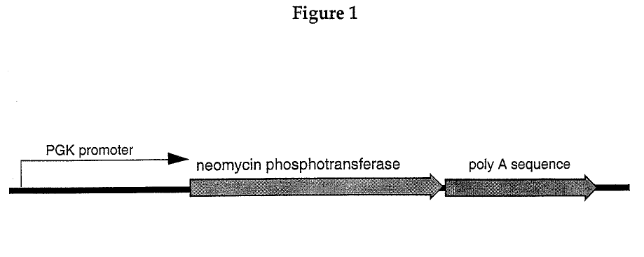

Conventional targeting vectors engineered for insertion of transgenes at selected sites (chromosomal loci) in the genome of interest consist of a 5′ homology arm, followed by the transgene of interest (frequently preceded by a particular promoter), a positive selection marker gene-containing cassette, and a 3′ homology arm. The selection marker gene-containing cassette used in these methods consists of a ubiquitously expressed promoter such as the phosphoglycerate kinase promoter which drives the expression of a positive drug selection gene such as neomycin phosphotransferase or other suitable drug selection gene familiar in the art, followed by a polyadenylation signal sequence to confer efficient polyadenylation of the transcribed message (FIG. 1). Since this selection cassette carries its own promoter, it confers drug resistance independent of whether it integrates at the desired (targeted) site (via homologous recombination) or at another site or sites (as a result of random/illegitimate recombination). Since integration of the cassette via homologous recombination into a target locus is a relatively rare event, many drug-resistant clones have to be screened to determine exactly which clones are correctly targeted (i.e. those clones in which the selection cassette has inserted at the chromosomal locus of choice as a result of specific homologous recombination) and which clones are not targeted (i.e. those clones in which the selection cassette has integrated randomly into the genome). Although some chromosomal loci can be targeted at a higher frequency than others, in general the screening process typically involves screening more than 100 clones by Southern blotting, PCR, or other standard method. These processes can be tedious, time-consuming, and costly.

Several approaches have been utilized to increase the frequency of targeted over non-targeted homologous recombination events or decrease the background, thus enabling easier detection of correctly targeted cells. One approach which decreases the background involves positive/negative selection, and it employs, in addition to the drug-resistance marker that can be selected for (positive selection drug resistance gene), a negative selection marker that can be selected against. An example of such a marker gene is herpes simplex virus (HSV) thymidine kinase, which can be selected against using gangcyclovir. In targeting vectors where the selection cassette employs positive/negative selection, the negative selection cassette is placed outside of the homology arms of the vector. Although there is not a large enough number of ‘side-by-side’ comparisons evaluating the efficiency of targeting achieved using the same homology arms but comparing using only positive versus positive/negative selection, it has been reported that positive/negative selection increases the representation of correctly targeted clones by approximately 5 to 10 fold over that which is achieved by the corresponding targeting vector utilizing only positive selection. One of the drawbacks of positive/negative selection, and also one of the reasons why it is not 100% efficient, is that the negative selection cassette can be inactivated by mutation or, more commonly, by methylation and, therefore, will not work, consequently allowing integration to occur at random sites. In addition, while it does reduce the number of clones that have to be screened, it does not completely alleviate the need for screening for correctly targeted events (Joyner, 1999, The Practical Approach Series, 293).

Another approach that has been used is called “exon trapping technology” which relies on engineering selection cassettes lacking a promoter. The selection cassettes typically used for exon trapping consist of a splice acceptor (SA) followed by the drug selection marker and a polyadenylation signal. When used to trap exons, this selection cassette is introduced into cells and allowed to insert randomly into the genome. Since the drug selection marker lacks its own promoter, it will only be expressed if it integrates downstream of an exon in a transcriptionally active gene. Both of these conditions (insertion within a transcriptionally active locus and insertion after an exon in that locus) must be met for the cell clone that carries the insertion to be resistant to the drug selection process. This type of selection strategy has been used to identify genes that are expressed in ES cells (Friedrich and Soriano, 1991, Genes Dev, 5,1513-23; Wiles et al., 2000, Nat Genet, 24,13-4). However, this selection strategy has not been routinely employed when engineering targeting vectors for several reasons including (1) it can only be used for genes that are expressed in ES cells; and (2) even in that application, it is considered “a method of last resort” because of the risk of selecting for differentiated ES cells. This arises if the gene is not normally expressed in undifferentiated ES cells. By selecting for drug resistance gene expression to be driven by a promoter of a locus that is not normally expressed in ES cells, one inadvertently selects for differentiated cells that express the targeted locus.

In accordance with the present invention, Applicants have combined for the first time: (1) targeting into a transcriptionally active locus, with (2) the use of a “promoter-less selection cassette” to effectively select for only those cells that are correctly targeted by utilizing a targeting vector that relies on the endogenous promoter of the locus being targeted for transcription of the drug selection gene. Because random insertion of a promoter-less drug selection marker very rarely leads to expression of that marker as result of insertion downstream of a transcriptionally active promoter, when such a cassette is directed through the use of homology arms to a specific transcriptionally active locus, essentially all of the resulting drug-resistant cells arise from homologous recombination between the targeting vector and the targeted locus. Thus, a targeting frequency of nearly 100% is obtained. In addition to having all the advantages of targeting engineered loci into a specific chromosomal locus, the novel technology described herein results in several important advances in the field of generating transgenic animals, including:

(a) It selects only for correctly targeted cells, leading to nearly 100% targeting frequency, therefore alleviating the need to screen for correctly targeted cells.

(b) Selecting only correctly targeted cells not only conserves time, labor, and cost, but also allows for the use of pools of drug-resistant targeted cells instead of individual cell clones for deriving transgenic animals.

(c) The use of pools of targeted cells instead of individual clones decreases the possibility that a transgenic animal is derived using a mutant clone or that chimeric animals derived from a clone will not transmit to the germ line.

The description and examples presented infra are provided to illustrate the subject invention. One of skill in the art will readily recognize that these examples are provided by way of illustration only and are not included for the purpose of limiting the invention.

### EXAMPLES

Many of the techniques used to construct DNA vectors described herein are standard molecular biology techniques well known to the skilled artisan (see e.g., Sambrook, J., E. F. Fritsch And T. Maniatis. Molecular Cloning: A Laboratory Manual, Second Edition, Vols 1, 2, and 3, 1989; Current Protocols in Molecular Biology, Eds. Ausubel et al., Greene Publ. Assoc., Wiley Interscience, NY). All DNA sequencing is done by standard techniques using an ABI 373A DNA sequencer and Taq Dideoxy Terminator Cycle Sequencing Kit (Applied Biosystems, Inc., Foster City, Calif.).

### Example 1

The following is a non-limiting example of the novel technology described herein. A DNA targeting vector was constructed consisting of an approximately 2 kb 5′ homology arm containing sequence downstream of exon 1 of the ROSA26 locus. The ROSA26 locus encodes for an RNA that is not translated into a protein. (It should be noted that if a transcriptionally active locus where exon 1 is translated, the promoter-less selection marker should be targeted at or before exon 1 or as a fusion to the protein normally encoded by the targeted locus). The 5′ homology arm extends from the NotI site to the NheI site (Friedrich and Soriano, 1991, Genes Dev, 5, 1513-23.; Soriano, 1999, Nat Genet, 21, 70-1.). A selection cassette was inserted at that site. The selection cassette in this specific example is SA-loxP-EM7-neo-4xpolyA-loxP, wherein SA is a splice acceptor sequence, the two loxP sites are the locus of recombination sites derived from bacteriophage P1 (Abremski and Hoess, 1984, J Biol Chem, 259, 1509-14), EM7 is a prokaryotic constitutively active promoter, neo is the neomycin phosphotransferase gene (Beck et al., 1982, Gene, 19, 327-36), and 4xpolyA is a polyadenylation signal engineered by linking in tandem the polyadenylation signal of the murine pgk gene (Adra et al., 1987, Gene, 60, 65-74) and three copies of a 254 bp BamHI fragment containing both early and late polyadenylation signals of Simian Virus 40 (SV40) (Reddy et al., 1978, Science, 200, 494-502; Thimmappaya et al., 1978, J Biol Chem, 253, 1613-8). The skilled artisan will recognize that many of the individual components in the selection cassette can be substituted with comparable components. For example, the loxP recombination sites can be substituted with FRT or other sites recognized by recombinases, the EM7 promoter can be substituted with any bacterial promoter that is silent in mammalian cells, and the neo gene can be substituted with any suitable selectable marker gene that can be selected for both in bacteria and in mammalian cells (Joyner, 1999, The Practical Approach Series, 293). After the second loxP site, an open reading frame (ORF) encoding for LacZ has been engineered followed by a β-globin polyadenylation signal (β-globin polyA) of the rabbit β-globin gene (ACCESSION K03256 M12603). Again, the skilled artisan will recognize that any ORF can be placed here in place of LacZ, depending on the desired result, and that other polyadenylation signals can be used in place of the β-globin polyA. The β-globin polyA is followed by a 3′ homology arm containing sequence that is continuous with the 5′ homology arm in the native ROSA26 locus. The 3′ homology arm extends approximately 9.4 kb past the site of insertion of the selection cassette and contains ROSA26 sequence up to the unique EcoRI site. The choice of what segment and how much of the locus sequence to include in the homology arm generally needs to be empirically determined. However, care should be taken not to include the promoter of the locus being targeted as part of the homology arms, as doing so would counteract the selection strategy. Note the absence of a mammalian promoter in the selection cassette and the use of a prokaryotic promoter, EM7. The EM7 promoter is silent in mammalian cells but can be used to drive neo expression in bacteria and thus confer the host E. coli with kanamycin resistance. In addition, this targeting vector contains an origin of replication and a β-lactamase gene, used to confer ampicillin resistance in host bacteria. Since the selection marker contained in this targeting vector lacks a mammalian promoter, the only way that this targeting vector can confer drug resistance to mammalian cells is if the selection marker integrates in appropriate fashion within a gene that is expressed in the target cell. The likelihood of this happening randomly is rather low since each cell type only transcribes a subset of all the genes in a genome. Thus, by including the 5′ and 3′ homology arms derived from the ROSA26 locus, Applicants are effectively and efficiently biasing for proper insertion of the targeting vector into the target locus. Subsequent to construction, the DNA targeting vector was introduced it into ES cells by standard methods familiar in the art and the percentage of targeting events was determined. Briefly, the targeting vector was linearized after the 3′ end of the 3′ homology arm by restriction enzyme digestion and transfected into ES cells employing standard methodology (Joyner, 1999, The Practical Approach Series, 293) and G418-resistant clones were selected, again by standard methods familiar in the art. Individual clones were picked and analyzed by standard Southern blotting to determine which clones were targeted. All clones examined were found to be correctly targeted.

To demonstrate the reproducibility and general applicability of the methods of the invention, equivalent DNA targeting vectors were constructed using the ORF cDNAs encoding for other genes (these genes essentially replaced lacZ in the vector described supra). Table 1 lists the targeting frequencies obtained using these DNA targeting vectors. Note that in the targeting vectors only the gene of interest is replaced. In these examples, the selection marker and other features of the DNA targeting vector remain the same.

TABLE 1

| | Number of G418-resistant | |
|---|---|---|
| transgene | clones screened | Targeting Frequency |
| (neo) | 8 | 100% (8/8) |
| hROR1 | 20 | 100% (20/20) |
| CMVp-lacZ | 14 | 100% (14/14) |
| hROR2 | 6 | 100% (6/6) |
| α1p-OGH | 7 | 100% (7/7) |
| SM22a-lacZ | 5 | 100% (5/5) |
| m(HTKL)2-Fc | 10 | 100% (10/10) |
| mMdk2-Fc | 8 | 100% (8/8) |
| SM22a-lacZ | 5 | 100% (5/5) |

We claim,

1. A method of targeting a promoter-less selection cassette into the ROSA26 locus in eukaryotic cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into eukaryotic cells;
c) selecting the eukaryotic cells of (b) for drug-resistance, and
d) screening the drug-resistant eukaryotic cells of (c) to identify those cells in which the promoter-less selection cassette has integrated by homologous recombination into the ROSA26 locus.

2. A method of targeting a promoter-less selection cassette into the ROSA26 locus in stem cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into stem cells;
c) selecting the stem cells of (b) for drug-resistance, and
d) screening the drug-resistant stem cells of (c) to identify those cells in which the promoter-less selection cassette has integrated by homologous recombination into the ROSA26 locus.

3. A method of targeting a promoter-less selection cassette into a ROSA26 locus in embryonic stem cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into embryonic stem cells;
c) selecting the embryonic stem cells of (b) for drug-resistance, and
d) screening the drug-resistant embryonic stem cells of (c) to identify those cells in which the promoter-less selection cassette has integrated by homologous recombination into the ROSA26 locus.

4. A method of targeting a promoter-less selection cassette into a transcriptionally active locus in eukaryotic cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into eukaryotic cells;
c) selecting the eukaryotic cells of (b) for drug-resistance, and
d) screening the drug-resistant eukaryotic cells of (c) to identify those cells in which the promoter-less selection cassette has integrated by homologous recombination into the transcriptionally active locus.

5. A method of targeting a promoter-less selection cassette into a transcriptionally active locus in stem cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into stem cells;
c) selecting the stem cells of (b) for drug-resistance, and
d) screening the drug-resistant stem cells of (c) to identify those cells in which the promoter-less selection cassette has integrated by homologous recombination into the transcriptionally active locus.

6. A method of targeting a promoter-less selection cassette into a transcriptionally active locus in embryonic stem cells, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into embryonic stem cells;
c) selecting the embryonic stem cells of (b) for drug-resistance, and
d) screening the drug-resistant embryonic stem cells of (c) to identify those cells in which the promoter-less selection cassette has integrated by homologous recombination into the transcriptionally active locus.

7. A method of genetically modifying a eukaryotic cell by targeting a promoter-less selection cassette into the ROSA26 locus, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into eukaryotic cells;
c) selecting the eukaryotic cells of (b) for drug-resistance, and
d) screening the drug-resistant eukaryotic cells of (c) to identify those cells in which the promoter-less selection cassette has integrated by homologous recombination into the ROSA26 locus.

8. A method of genetically modifying a stem cell by targeting a promoter-less selection cassette into the ROSA26 locus:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into stem cells;
c) selecting the stem cells of (b) for drug-resistance, and
d) screening the drug-resistant stem cells of (c) to identify those cells in which the promoter-less selection cassette has integrated by homologous recombination into the ROSA26 locus.

9. A method of genetically modifying an embryonic stem cell by targeting a promoter-less selection cassette into a ROSA26 locus, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into embryonic stem a cells;
c) selecting the embryonic stem cells of (b) for drug-resistance, and
d) screening the drug-resistant embryonic stem cells of (c) to identify those cells in which the promoter-less selection cassette has integrated by homologous recombination into the ROSA26 locus.

10. A method of genetically modifying a eukaryotic cell by targeting a promoter-less selection cassette into a transcriptionally active locus, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into eukaryotic cells;
c) selecting the eukaryotic cells of (b) for drug-resistance, and
d) screening the drug-resistant eukaryotic cells of (c) to identify those cells in which the promoter-less selection cassette has integrated by homologous recombination into the transcriptionally active locus.

11. A method of genetically modifying a stem cell by targeting a promoter-less selection cassette into a transcriptionally active locus, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into stem cells;
c) selecting the stem cells of (b) for drug-resistance, and
d) screening the drug-resistant stem cells of (c) to identify those cells in which the promoter-less selection cassette has integrated by homologous recombination into the transcriptionally active locus.

12. A method of genetically modifying an embryonic stem cell by targeting a promoter-less selection cassette into a transcriptionally active locus, comprising:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into embryonic stem cells;
c) selecting the embryonic stem cells of (b) for drug-resistance, and
d) screening the drug-resistant embryonic stem cells of (c) to identify those cells in which the promoter-less selection cassette has integrated by homologous recombination into the transcriptionally active locus.

13. The method of claim 7, 8, 9, 10, 11, or 12, wherein the genetic modification to the transcriptionally active locus comprises deletion of a coding sequence, gene segment, or regulatory element; alteration of a coding sequence, gene segment, or regulatory element; insertion of a new coding sequence, gene segment, or regulatory element; creation of a conditional allele; or replacement of a coding sequence or gene segment from one species with an homologous or orthologous coding sequence from the same or a different species.

14. The method of claim 13, wherein the alteration of a coding sequence, gene segment, or regulatory element comprises a substitution, addition, or fusion.

15. The method of claim 14, wherein the fusion comprises an epitope tag or bifunctional protein.

16. The method of claim 3, 6, 9, or 12, wherein the embryonic stem cell is a mouse, rat, or other rodent embryonic stem cell.

17. A non-human organism containing a genetically modified ROSA26 locus, produced by a method comprising the steps of:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the ROSA26 locus;
b) introducing the DNA targeting vector of (a) into eukaryotic cells;
c) selecting the eukaryotic cells of (b) for drug-resistance,
d) screening the drug-resistant eukaryotic cells of (c) to identify those cells in which the promoter-less selection cassette has integrated by homologous recombination into the ROSA26 locus,
e) introducing the eukaryotic cells of (d) into a blastocyst; and
f) introducing the blastocyst of (e) into a surrogate mother for gestation.

18. A non-human organism containing a genetically modified transcriptionally active locus, produced by a method comprising the steps of:
a) constructing a DNA targeting vector containing a nucleotide sequence, comprising:
a 5′ homology arm,
a promoter-less selection cassette, and
a 3′ homology arm,
wherein the promoter-less selection cassette is comprised of a promoter-less selectable marker gene, a gene of interest, and a polyadenylation signal sequence and wherein the 5′ and 3′ homology arms are derived from the transcriptionally active locus;
b) introducing the DNA targeting vector of (a) into eukaryotic cells;
c) selecting the eukaryotic cells of (b) for drug-resistance,
d) screening the drug-resistant eukaryotic cells of (c) to identify those cells in which the promoter-less selection cassette has integrated by homologous recombination into the transcriptionally active locus,
e) fusing the eukaryotic cell of (d) with another eukaryotic cell; and
f) introducing the fused eukaryotic cell of (e) into a surrogate mother for gestation.

19. The non-human organism of claim 17 or 18, wherein the genetic modification to the transcriptionally active locus comprises deletion of a coding sequence, gene segment, or regulatory element; alteration of a coding sequence, gene segment, or regulatory element; insertion of a new coding sequence, gene segment, or regulatory element; creation of a conditional allele; or replacement of a coding sequence or gene segment from one species with an homologous or orthologous coding sequence from the same or a different species.

20. The non-human organism of claim 19, wherein the alteration of a coding sequence, gene segment, or regulatory element comprises a substitution, addition, or fusion.

21. The non-human organism of claim 20, wherein the fusion comprises an epitope tag or bifunctional protein.

22. The non-human organism of claim 17 or 18, wherein the eukaryotic cell is a stem cell.

23. The non-human organism of claim 22, wherein the stem cell is an embryonic stem cell.

24. The non-human organism of claim 23, wherein the embryonic stem cell is a mouse, rat, or other rodent embryonic stem cell.

25. The non-human organism of claim 17 or 18, wherein the blastocyst is a mouse, rat, or other rodent blastocyst.

26. The non-human organism of claim 17 or 18, wherein the surrogate mother is a mouse, rat, or other rodent.

27. The non-human organism of claim 17 or 18, which is a mouse.
